(19) United States
(12) Patent Application Publication
(10) Pub. No.: US 2020/0075157 A1
(43) Pub. Date: Mar. 5, 2020

(54) HEALTHCARE TRACKING SYSTEM TO IMPROVE SERVICE SATISFACTION, COMMUNICATION, AND PATIENT EXPERIENCE

(71) Applicant: Shelley Monheiser, Edwardsville, IL (US)

(72) Inventor: Shelley Monheiser, Edwardsville, IL (US)

(21) Appl. No.: 16/552,900

(22) Filed: Aug. 27, 2019

Related U.S. Application Data
(60) Provisional application No. 62/724,131, filed on Aug. 29, 2018.

(51) Int. Cl. G16H 40/20; G16H 70/20

(52) U.S. Cl. CPC G16H 40/20; G16H 70/20

(57) ABSTRACT

A healthcare tracking system and method identifies an electronic mobile device of a healthcare facility patient and/or a patient-selected approved person. A current state of a healthcare workflow involving administration of healthcare services to the patient at the healthcare facility is determined, and the electronic mobile device is directed to display information representative of the current state of the healthcare workflow for the patient. One or more changes to the current state of the healthcare workflow are determined, and the electronic mobile device is directed to update the information that is displayed to correspond with the one or more changes to the current state of the healthcare workflow.

### CROSS-REFERENCE TO RELATED APPLICATIONS

This application claims priority to U.S. Provisional Application No. 62/724,131, which was filed on 29 Aug. 2018, and the entire disclosure of which is incorporated herein by reference.

### BACKGROUND

Healthcare facilities treat tens of millions of patients each year. This number continues to grow with longer life expectancies and growing populations. The increasing workload on healthcare facilities places increasing burdens on medical staff and personnel. These increasing burdens can delay the provision of services to patients, and can interfere with clear and timely communication with patients and families of patients.

For example, a person seeking treatment at an emergency room usually is initially triaged and then may wait for an emergency room assignment depending on the urgency of the person's medical condition. Once assigned to a room, the patient waits for visits by nurses and doctors, which may order various tests or examinations to be performed on the patient to diagnose the medical problem of the patient. The patient then waits for other personnel to perform the tests or examinations. Depending on the available staff and current patient workload, there can be significant delays in completing the tests. Many times, the facility staff does not communicate the status of the examination of the patient, including the status of tests, what additional tests are to be performed, whether the patient will be admitted to the hospital, what remaining steps are required to be performed before the patient is discharged, etc., in a timely manner. As a result, the patient and family of the patient are left wondering whether and when the patient will be able to leave the hospital.

Many healthcare facilities receive financial awards or fines based on the level of patient satisfaction with the care received at the facilities. Due to the poor communication of the overall treatment plan and status of the plan progress to patients, patients often express significant dissatisfaction with healthcare facilities. As a result, many healthcare facilities face stiff fines or miss out on financial awards due to poor communication with patients and the families of patients.

### BRIEF DESCRIPTION

In one embodiment, a method is provided that includes identifying an electronic mobile device of one or more of a patient at a healthcare facility or at least one approved person selected by the patient, determining a current state of a healthcare workflow involving administration of healthcare services to the patient at the healthcare facility, directing the electronic mobile device to display information representative of the current state of the healthcare workflow for the patient, identifying one or more changes to the current state of the healthcare workflow, and directing the electronic mobile device to update the information that is displayed to correspond with the one or more changes to the current state of the healthcare workflow.

In one embodiment, a system includes one or more processors of a communication controller configured to identify an electronic mobile device of one or more of a patient at a healthcare facility or at least one approved person selected by the patient. The one or more processors also are configured to determine a current state of a healthcare workflow involving administration of healthcare services to the patient at the healthcare facility, and to direct the electronic mobile device to display information representative of the current state of the healthcare workflow for the patient. The one or more processors also are configured to identify one or more changes to the current state of the healthcare workflow, and to direct the electronic mobile device to update the information that is displayed to correspond with the one or more changes to the current state of the healthcare workflow.

### BRIEF DESCRIPTION OF THE DRAWINGS

The present inventive subject matter will be better understood from reading the following description of non-limiting embodiments, with reference to the attached drawings, wherein below:

FIG. 1 illustrates one embodiment of a healthcare tracking system;

FIG. 2 illustrates a flowchart of a method for tracking and communication progression of a healthcare workflow;

FIG. 3 illustrates one example of a graphical user interface (GUI) that can be presented on a mobile electronic device by the tracking system;

FIG. 4 illustrates one example of another GUI that can be presented on a mobile electronic device by the tracking system;

FIG. 5 illustrates one example of another GUI that can be presented on a mobile electronic device by the tracking system;

FIG. 6 illustrates one example of another GUI that can be presented on a mobile electronic device by the tracking system;

FIG. 7 illustrates one example of another GUI that can be presented on a mobile electronic device by the tracking system;

FIG. 8 illustrates one example of another GUI that can be presented on a mobile electronic device by the tracking system;

FIG. 9 illustrates one example of another GUI that can be presented on a mobile electronic device by the tracking system;

FIG. 10 illustrates one example of another GUI that can be presented on a mobile electronic device by the tracking system;

FIG. 11 illustrates one example of another GUI that can be presented on a mobile electronic device by the tracking system;

FIG. 12 illustrates one example of another GUI that can be presented on a mobile electronic device by the tracking system;

FIG. 13 illustrates one example of another GUI that can be presented on a mobile electronic device by the tracking system; and

FIG. 14 illustrates one example of a GUI presented on a mobile electronic device that notifies a healthcare provider of a lab result.

### DETAILED DESCRIPTION

The inventive subject matter described herein relates to communication systems and methods that keeps interested parties informed and involved in the progression of healthcare activities administered on a person. Due to the rising cases of diseases, infections and complications, the lack of medical education to patients, lack of understanding care, and lack of overall communication and continuity has amounted in hefty governmental fines being imposed on healthcare facilities in an effort to increase patient satisfaction. The increasing emergency room utilization, human capital and cost to operate has left some hospitals in financial ruin. Overcrowding has amounted to long wait times, increased patient risk, overworked and stressed emergency department staff members with not enough time with increased patient loads to effectively and efficiently communicate all necessary information to all patients. Patients wait for an evaluation for hours without routine checks as they feel they are ignored or forgotten by the triage staff, or they recognize the volume of patients also waiting and they don't want to interrupt the staff.

The inventive subject matter described herein changes how healthcare communication is established and maintained; both between patient and provider as well as provider to provider. It will assist the provider in delivering accurate, efficient care and will allow for easy accessibility to the patient and their designated representatives.

The systems and methods track and update the status of a person receiving medical treatment, and provide this information to interested parties, such as family members, doctors, nurses, or the like. Use of the systems and methods can improve relations with and satisfaction of persons receiving treatment because these persons are kept up-to-date with the progression of clinical workflow being performed on the persons. The system and method can keep patients, families, and provider care teams informed of ordered, in-process, and completed testing while a patient is seeking medical (e.g., emergency) treatment. The system and method also will allow for automated disposition and follow up communication to be provided to the patients' inclusive group. As one example, the systems and methods can track registration of a patient in an emergency room facility, as well as the tests or examinations needed to be performed on the patient before a decision on whether to discharge the patient from the facility or to admit the patient into a hospital for an extended stay. The systems and methods can update the patient and/or family members as to how much longer it will be before the patient is examined by a healthcare provider (e.g., a doctor or nurse), how much longer it will be before the patient is to be taken to a room for a test or examination, how much longer it will be before the patient is expected to be discharged or admitted, and the like.

The system and method can send a communication to a healthcare team responsive to completion of testing ordered for the patient and when the test results and relevant data can be collected to accurately decipher turnaround times to both establish and maintain value-based purchasing metrics. Patient safety will increase from use of this system and method as patients are able to use the system and method for communication while care is actively provided to the patients, as well as after discharge of the patients. This will allow for continuity of care, proper education, and appropriate follow up as instructed to the patient but that the patient may not remember during an emergent event.

Often times, primary care providers are not informed when their patient arrives to the hospital for emergency care. The system and method can notify the primary care physician when identified by the patient or another party upon patient arrival to the emergency department of a hospital, an urgent care facility, another health clinic, or another outpatient setting or facility. The primary care provider can then access the system and method to follow the care provided to the patient while the patient is in the outpatient setting, to communicate with an emergency room provider, to help reduce re-admissions of patients if primary care has been in recent contact with patient, help establish a plan of care in conjunction with an educational or emergency department team, etc.

Keeping the patient, family members of the patient, and/or healthcare providers updated with the status and progression of the patient through providing healthcare and making healthcare decisions can significantly improve the experience of the patient at a healthcare facility. A significant complaint or source of dissatisfaction of patients is the lack of communication between the healthcare providers or facility and the patients regarding the status of the patient and upcoming decisions to be made. This can cause significant frustration to patients and family members of the patient. The system and method can keep family members informed of the current status of a patient that is usually “behind the scenes” and not otherwise known by the family members.

One impact of patient dissatisfaction on healthcare facilities can be the loss of financial awards, the reduction of payments, and/or the imposition of fines or other penalties on the facilities. For example, those facilities having greater patient dissatisfaction may receive fewer financial awards, be paid less by insurance companies, and/or may be fined by government entities or other entities. Improving the communication of the patient status and progression through a healthcare workflow and decision-making progress will improve patient satisfaction, thereby increasing the financial compensation to and/or reducing the financial penalties levied on healthcare facilities.

While the description herein focuses on patients being treated in hospitals, not all embodiments of the inventive subject matter are limited to hospitals or human patients. For example, one or more embodiments of the inventive subject matter can be used to improve communication with persons or other interested parties for persons receiving care or treatment in outpatient facilities, other inpatient facilities, urgent care facilities, veteran hospitals, nursing homes, assisted living facilities, schools or other educational institutions, health clinics, school athletic facilities, or the like. Optionally, at least one embodiment can be used to improve communication with persons or interested parties for animals or pets receiving care or treatment at veterinarian hospitals, clinics, or facilities. The information described herein can be communicated to patients or other interested parties, such as healthcare providers, family members, friends, or the like, who are selected and/or legally permitted to have access to and/or be informed of medical information related to the person. For example, a patient can select those persons that can receive medical information about the patient in compliance with the Health Insurance Portability and Accountability Act (HIPAA). These persons can be provided with updates on the progression and status of the healthcare workflow for the patient.

In one embodiment, the systems and methods described herein can provide the person receiving healthcare with the ability to express their satisfaction or dissatisfaction with the healthcare received and/or with the healthcare facility using the same mobile device that was used to update the patient and/or interested persons with updates on the progression and status of the healthcare workflow for the patient. For example, a survey or questionnaire can be provided to the patient soon after treatment is completed via a mobile device. This can allow for the patient or interested persons to express their satisfaction or dissatisfaction soon after receiving care. With the increased communication provided by the systems and methods described herein, the patients and interested persons are more likely to express satisfaction with the received care than without the increased communication or with delaying communication of the survey or questionnaire to the patient or persons.

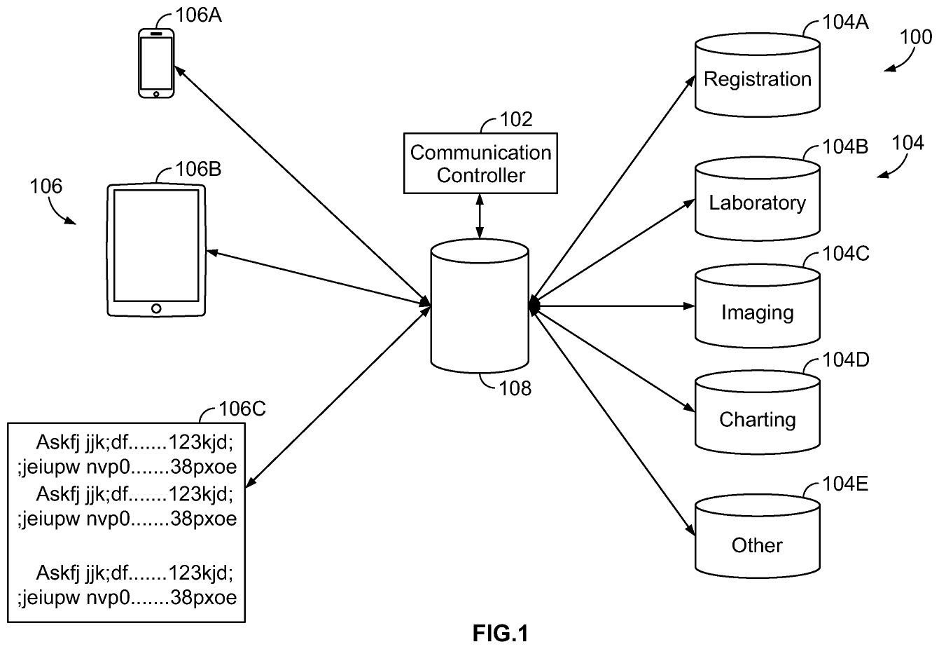

FIG. 1 illustrates one embodiment of a healthcare tracking system 100. The tracking system 100 optionally can be referred to as a communication system in that the tracking system 100 communicates the status and progression of a healthcare workflow to a person receiving care and/or interested parties. The tracking system 100 includes a communication controller 102 that directs the communication of information between, to, and/or from several data source devices 104 and electronic devices 106. The electronic devices 106 represent output devices through which persons can receive or view statuses and/or progressions of healthcare workflows, such as a mobile phone 106A, a tablet computer 108B, another display device 108C, or the like. The communication controller 102 represents hardware circuitry that includes and/or is connected with one or more processors (e.g., one or more integrated circuits, one or more field programmable gate arrays, one or more microprocessors, or the like) that perform the operations described herein in connection with the controller 102. The electronic devices 106 can operate using one or more software applications that receive, retrieve, and/or display information as permitted by the communication controller 102. In one embodiment, the electronic devices 106 can integrate directly with electronic medical record software and receive feeds from electronic medical records in a live format or manner.

The data source devices 104 represent one or more devices that provide, store, generate, or otherwise allow access to information data related to the healthcare workflow associated with a patient. The data source devices 104 can represent input devices (e.g., keyboards, touchscreens, microphones, etc.), sensors, tangible and non-transitory computer readable storage media (e.g., computer memories, such as hard drives, servers, removable drives, optical discs, etc.), computers, or the like.

One example of a data source device 104 is a registration database or registration input device 104A. The registration device 104A can generate and/or store data indicative of information on whether various persons are in the healthcare facility, whether the persons have been admitted for one or more overnight stays in the healthcare facility, whether the persons have been discharged from the facility, when the persons are expected or estimated to be admitted, when the persons are expected or estimated to be discharged, steps or operations that need to be completed before the persons are admitted or discharged, and the like. Other information that can be stored in the registration device 104A includes the room in which the person is admitted, is being discharged from, or is located. The registration device 104A also can store information on which other persons can contact (e.g., call), visit, etc., the patient. For example, the registration device 104A can store identifications of which persons that a patient has identified as being permitted to receive confidential medical information about the patient. The registration device 104A can store information identifying the doctor that admitted the patient, the phone number of the room where the patient is admitted, the names of the nurses that will be treating the patient, and the like. In one embodiment, the registration device 104A can store information identifying a case manager associated with the patient. For example, the case manager can be identified and associated with the patient so that the case manager can track progress of treatment of the patient. The system and method can begin sending notifications to the mobile device of the case manager once a defined event occurs, such as the patient being in the health care facility and receiving treatment for more than a designated period of time (e.g., twenty-four hours), a test taking longer than another designated period of time to complete, the patient waiting to see a particular doctor or other healthcare provider for longer than another period of time, etc.

The information stored in the registration device 104A can be automatically generated based on associations between data stored in other devices 104. For example, the registration device 104A can store an estimate of when a person will be discharged from a hospital that is based on how many steps need to be performed before discharge can occur. These steps can include completion of an examination by a doctor, receiving input from a doctor or nurse that the person can be discharged, a current location or status of a nurse that is to assist the person with leaving the facility, when the nurse will be available to assist the person with leaving the facility (e.g., when the nurse will be done working with another person or arrive at the facility), etc. Optionally, the information stored in the registration device 104A can be input by one or more operators (e.g., nurses, registration officials, etc.).

The devices 104 can include one or more laboratory databases or input devices 104B that generates and/or stores data containing information on tests or examinations that have been ordered by healthcare personnel, the statuses of the tests or examinations, the results of the test or examinations, and the like. For example, the laboratory device 104B can store a type of blood test that a doctor ordered, indications of whether blood of a patient has been obtained for the test, whether the blood has been examined or tested, and/or the results of the test. The devices 104 can include an imaging database or input device 104C. The device 104C generates and/or stores data containing information on orders for imaging examinations to be performed, the results or diagnoses from examining images, the status of imaging examinations, wait times for an imaging system to be available, and the like. The imaging examinations can include, by way of non-limiting example, ultrasound exams, CT exams, MM exams, and the like.

The devices 104 can include one or more charting database or input devices 104D. These devices 104D can generates and/or stores charting data containing information on notes about a patient from a healthcare provider, test results, diagnoses, symptoms, treatment procedures, and the like. The healthcare providers can input the information into the devices 104D for storage and later retrieval. The devices 104 can include one or more other databases or input devices 104E that generate and/or store data containing other information on patients, healthcare provided to the patients, the workforce of healthcare personnel, and/or statuses of the healthcare facility. For example, information on wait times for tests to be completed in the laboratory, wait times for examinations to be completed by doctors, changes in work shifts by healthcare personnel, and the like, can be generated and/or stored in the devices 104E.

Data that are generated and/or stored in the devices 104 can be stored in one or more aggregate healthcare databases 108. The healthcare database 108 can represent one or more tangible and non-transitory computer readable media that receive and/or obtain data collected, generated, and/or stored in the devices 104. For example, each device 104 may be used to create and/or store different types of data for the same or different patients in a healthcare facility. The laboratory device 104B may store test results on many patients, while the imaging device 104C can store the statuses of imaging examinations on some or all these same patients. But, the laboratory device 104B may not create or store data on the statuses of the imaging examinations, and the imaging device 104C may not create or store data on the test results. The data created and/or stored by or in the devices 104 can be aggregated in the healthcare database 108 to allow data from different sources for different patients in a healthcare facility to be collected and accessible. Different sets of data from different devices 104 for the same patient can be collected and stored in the database 108 so that the sets of data for the same patient are associated with a unique identification of that patient.

The controller 102 can control what information in the database 108 is communicated to or is able to be accessed by the devices 106. For example, the controller 102 can communicate with the devices 106 to determine whether the devices 106 are running software applications needed to access the data, and whether the devices 106 have been identified as being allowed to access one or more sets of the data associated with a patient. For a patient, the controller 102 can obtain identifications of which devices 106 are associated with persons that are allowed by the patient to view healthcare data for that patient. These identifications can be created and/or stored in the registration device 104A or another device. The controller 102 can then communicate the data in the database 108 that is associated with that patient to only those approved devices 106 associated with persons approved by the patient. Optionally, the controller 102 may only allow those approved devices 106 associated with persons approved by the patient to access the data in the database 108 that is associated with the patient.

The controller 102 can direct notifications be communicated to the devices 106 of persons associated with and/or approved by the patient as developments occur. For example, the controller 102 can begin sending notifications to the mobile device of the case manager once a defined event occurs, such as the patient being in the health care facility and receiving treatment for more than a designated period of time (e.g., twenty-four hours), a test taking longer than another designated period of time to complete, the patient waiting to see a particular doctor or other healthcare provider for longer than another period of time, etc. As another example, families of patients can be directly notified of both emergency room and inpatient dispositions when statuses are updated in the medical record of the patient. This can allow the families to plan for discharge of the patient or an extended stay of the patient while continuing to work or be home with family when applicable. Likewise, healthcare multidisciplinary team members can get live updates on the status of the patient as opposed to waiting for a one-to-one connection with a physician leading the care team to best utilize time and resources of the entire facility and team.

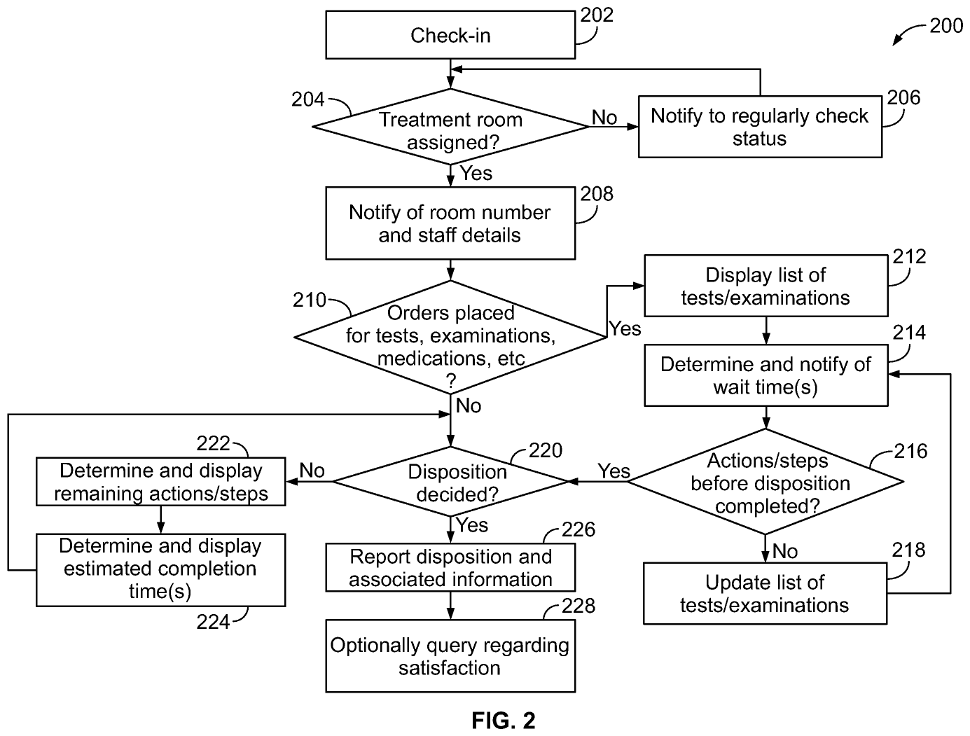

FIG. 2 illustrates a flowchart of a method 200 for tracking and communication progression of a healthcare workflow. The method 200 can represent operations performed by the tracking system 100 shown in FIG. 1. For example, the operations described in connection with the method 200 can be performed by the controller 102 and/or the devices 106. At 202, a patient checks in with the healthcare facility. During this process, a registrar can use the registration device 104A to obtain information from the patient, such as the name, birthday, symptoms, and the like, of the patient. Additionally, the registrar can obtain a list of persons that the patient approves to be notified of the healthcare workflow of the patient. This list can include unique identifications of or associated with the approved persons, such as mobile phone numbers of the devices 106, email addresses of the approved persons, or the like. The controller 102 can obtain the unique identifications of the approved persons or devices 106 from the registration device 104A, and can send software or links to obtain the software that is to be installed on the devices 106 to view and receive updates on the progression of the healthcare workflow of the patient. The devices 106 can then be used to obtain and install the needed software.

At 204, a determination is made as to whether the patient has been assigned to a treatment room at the healthcare facility. For example, the patient may have arrived at an emergency room or urgent care facility and may wait in a waiting room to be assigned to a room for further examination and/or treatment. The registration device 104A can generate or modify data in the device 104A and/or database 108 to indicate whether the patient has been assigned to and/or received in a treatment room at the facility. The controller 102 can examine this data associated with the patient to determine if the patient has been assigned to a treatment room.

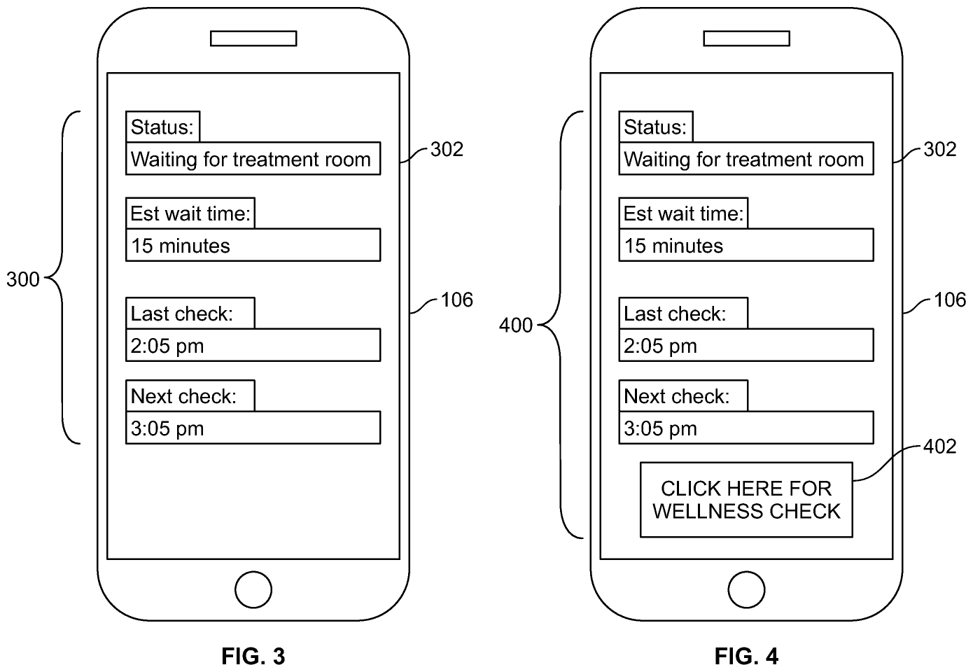

FIG. 3 illustrates one example of a graphical user interface (GUI) 300 of a device 106. The GUI 300 can be presented on a display screen 302 of the device 106 before the patient has been assigned to a treatment room. The controller 102 can direct the device 106 to present the information shown in FIG. 3 and/or can provide the device 106 with access to data stored in the database 108 containing the information shown in FIG. 3. As shown, the device 106 can present a status of the patient (“Waiting for treatment room”) based on data input into the registration device 104A. The device 106 also can present an estimated wait time before the patient is assigned to a treatment room. This time can be input into the registration device 104A, or can be calculated by the controller 102. For example, the controller 102 may estimate the wait time for a treatment room assignment based on how many treatment rooms are occupied, how long the different treatment rooms have been occupied, and an estimated time that patients are in treatment rooms before being discharged or admitted. This estimated time can be based on previous visits to the healthcare facility by the same or other patients, as well as the initial determinations of the health of the patient as determined by a triage of the patient upon check-in at the facility. Optionally, information associated with a patient can be accumulated and electronically tracked when staff assign themselves to the care team for the patient.

Primary care doctors, nursing homes, jails, any other outside agency often are not notified when a patient is admitted to a facility or requires outpatient follow up. The controller 102 can automatically send this information to included and necessary people and personnel. The controller 102 can link the inpatient care team with the primary care team for continuity of care and increased patient satisfaction and safety. The controller 102 also can communicate with pharmacies to obtain information on forgotten medications, medications that have been recently filled, medications that may have been missed, etc. Many times, patients do not remember all of their medications, families are unaware of medications and medication errors, overdosing or contraindications are missed due to lack of information and/or communication, and the like. Often times, especially in the elderly, multiple physicians and specialists care for the same patient without communicating. With the controller 102, all of the history, current treatment plans to include medications can be easily accessible and provided to the devices 106 by the controller 102.

EReturning to the description of the flowchart of the method 200 shown in FIG. 2, if the patient has not yet been assigned to a treatment room, then flow of the method 200 can proceed toward 206. Otherwise, if the patient has been assigned to a treatment room, then flow of the method 200 can proceed toward 208.

At 206, a status of the patient that has not yet been assigned to a treatment room is notified to be checked. For example, the device 106 of the patient may remind the patient to periodically go to a nurses' station, the registration device 104A, or to another location or person at the healthcare facility. The status of the patient can then be checked. This can prevent a sick or injured patient from being left untreated or unchecked for long periods of time during busy times at the healthcare facility when the wait times for a treatment room may be very long. As shown in the GUI 300 in FIG. 3, the device 106 can present information on when the last status check of the patient occurred, and when the next status check is due. The controller 102 can examine data input by the registration device 104A by a registrar, nurse, or other healthcare personnel to determine when the last status or wellness check on the waiting patient occurred. The controller 102 can instruct the device 106 to update the last and next status check times on the device 106 based on this data from the registration device 104A. If the status of the patient has not been checked before the next status check time, the controller 102 can send a signal to a device 106 carried by a healthcare personnel (e.g., a nurse, doctor, or the like) to go and check on the patient. This can ensure that a severely ill or injured patient is not forgotten about or neglected during busy time periods of a healthcare facility.

FIG. 4 illustrates another example of a GUI 400 of the device 106. The GUI 400 can present a selectable image, text, or icon 402 when a status check of the patient is due. The patient holding the device 106 can then select the image, text, or icon 402 to indicate that the patient is still conscious and/or that the status of the patient has not deteriorated. Responsive to this selection, the device 106 can notify the controller 102, which can store data in the database 108 indicating a successful status check. The device 106 can then update the information presented on the GUI 300 and/or 400 to update the last and next status checks.

Returning to the description of the flowchart of the method 200 shown in FIG. 2, flow of the method 200 can repeat through the operations of 204, 206 to repeatedly check on the status of a waiting patient until the patient is assigned to a treatment room. Once a patient is assigned to a treatment room, at 208, a notification is provided of the details of the assigned room and healthcare staff treating the patient. This notification can include a list or other structure of information presented on the device or devices 106 (e.g., of the patient and patient-selected persons) that includes the treatment room number or treatment room location, the name(s) of the healthcare personnel that will be examining or treating the patient (e.g., names of nurses and doctors), and the like. The controller 102 can direct the device(s) 106 of what information to present to the patient and/or approved persons. The notification optionally can include an estimated wait time before healthcare personnel will visit the patient in the treatment room. The controller 102 may estimate the wait time based on how many treatment rooms are occupied by other patients, how long the different treatment rooms have been occupied, and an estimated time that patients are in treatment rooms before visited by a healthcare personnel based on previous visits to the healthcare facility by the same or other patients.

The controller 102 can direct the device(s) 106 of the patient and/or approved persons to present a summary of a healthcare workflow for the patient. The healthcare workflow is a group or sequence of steps, operations, actions, etc., that are planned to be performed on or with the patient to treat the patient, diagnose one or more healthcare concerns of the patient, or the like, before the patient is discharged from the healthcare facility or admitted (or transferred) to another healthcare facility. For example, a healthcare workflow can include the steps that are selected by healthcare personnel to be performed before a patient is discharged from an emergency room or admitted to a room in a hospital. The workflow can include tests or examinations to be performed, medications to be administered to the patient, decisions to be made by healthcare personnel, or the like. The healthcare workflow can be dynamic in that additional steps or operations can be added to the workflow based on the outcome of other tests, examinations, decisions, or the like.

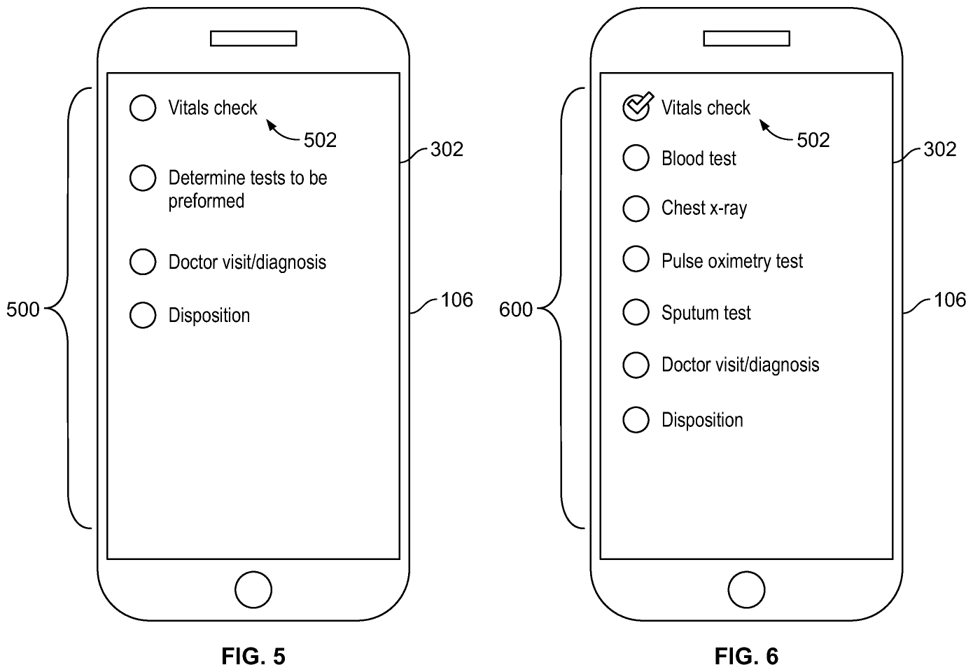

FIG. 5 illustrates one example of a GUI 500 presented on the device 106 that summarizes a healthcare workflow for the patient. As shown, the GUI 500 includes a list of actions 502 to be completed before the healthcare workflow is completed. The list shown in FIG. 5 is provided as one example of the different actions that may be involved in completing a workflow, but other actions may be included. The actions 502 may be presented in another manner, but are shown in a way that describes to the viewer of the device 106 of the sequence or order in which the actions 502 are to be completed (in one embodiment of the inventive subject matter). For example, the illustrated workflow requires that vitals of the patient be checked (“Vitals check,” such as checking the temperature, blood pressure, pulse, etc., of the patient), then determine which tests or examinations are to be performed, then a healthcare personnel visit the patient and/or made a diagnosis on the healthcare problem of the patient that brought the patient to the healthcare facility (“Doctor visit/diagnosis”), and finally, a disposition of the patient. This disposition can involve admitting the patient into a hospital, transferring the patient to another healthcare facility, or discharging the patient from the current healthcare facility.

The order in which the actions 502 in the healthcare workflow are performed or completed may differ from the order in which the actions 502 are listed or shown on the device 106. As shown and described herein, the controller 102 can direct the device 106 to update the list of actions 502 as various actions 502 are completed. This can clearly inform the patient and approved persons of the status of the healthcare workflow of the patient, as well as what actions 502 remain to be completed before the patient is admitted or discharged.

Returning to the description of the flowchart of the method 200 shown in FIG. 2, at 210, a determination is made as to whether any orders for the patient have been made. Healthcare personnel can request tests or examinations such as blood tests, imaging tests (e.g., x-ray images, MRI images, etc.), physical therapy, or the like. Healthcare personnel also can request that certain medications be provided to the patient, that other personnel examine the patient (e.g., a specialist in a particular medical field), or the like. The healthcare personnel can input these orders into one or more of the devices 104, such as the charting device 104D. The orders can be stored in the device(s) 104 and/or in the database 108, where the controller 102 can determine which orders have been placed.

If one or more tests, examinations, medications, etc. have been ordered, then flow of the method 200 can proceed toward 212. Otherwise, flow of the method 200 can proceed toward 220 if no tests, examinations, medications, or the like, have been ordered. At 212, the tests, examinations, medications, etc., that are ordered for the patient are presented on the device(s) 106. The controller 102 can direct the device(s) 106 to present information on the placed orders so that the patient and/or approved persons are apprised of which tests, examinations, medications, etc., have been requested for the patient.

For example, the list of actions 502 shown in FIG. 5 can be updated as shown in the updated GUI 600 shown in FIG. 6. The GUI 600 lists several tests or examinations to be performed, such as a blood test on the patient, an x-ray of the patient's chest be obtained, a pulse oximetry test be performed on the patient, and the sputum of the patient be examined (“Sputum test”). The tests that are listed on the GUI 600 can be determined by the controller 102 based on the results of the vitals check or an initial examination by a healthcare personnel. The list of tests can be updated based on the outcome of one or more actions 502. For example, if the outcome of the blood test reveals other potential medical issues, then the list of actions 502 can be updated to add additional tests and/or remove tests previously listed as actions 502 to be completed.

The controller 102 can obtain the results of the test as the tests are completed and/or the results are available (e.g., from the devices 104) and communicate these results to the devices 106. This can help ensure a higher level of quality patient care for time-sensitive diagnosis by giving live updates on critical labs or other test results. This allows the care team to continue bedside care all while being notified via the devices 106 of results that need attention. Currently, a healthcare provider may need to return to a computer workstation, refresh the screen and wait for updates that are usually batched and pushed every few minutes.

Additionally, the controller 102 can direct the device 106 to update the list of actions 502 to show that the vitals check action 502 has been completed, as shown in FIG. 6. The controller 102 can determine that the vitals check was completed based on data input into the charting device 104D by healthcare personnel and/or based on data saved to the database 108 by the charting device 104D. The GUI 600 shows the vitals check action 502 being completed by placing a checkmark in a circle associated with the vitals check action 502. Alternatively, the GUI 600 can show completion of actions 502 in another way, such as by changing the color or appearance of icons, text, etc., changing the length and/or color of a line showing progress of the healthcare workflow, or the like.

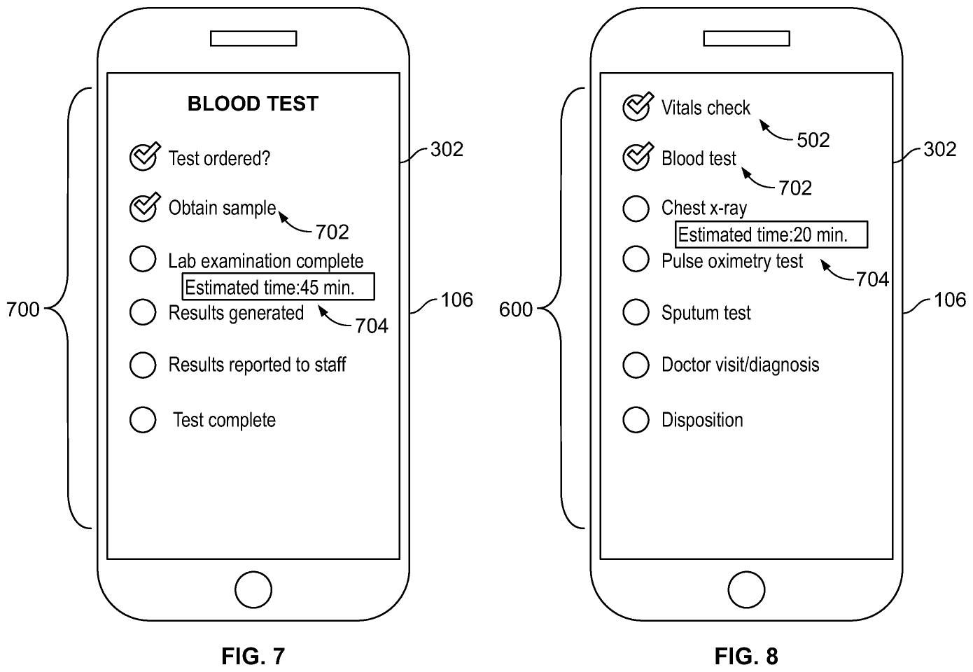

One or more of the actions 502 in the workflow may have a series of steps to complete the action. FIG. 7 illustrates another example of a GUI 700 that can be shown on the device(s) 106 to inform the patient or approved person(s) of the steps to be completed for completion of an action 502. In the illustrated example, the controller 102 directs the device 106 to display a list of steps 702 to be performed to complete the blood test action 502 shown in the GUI 600. These steps 702 are provided merely as one example, and include ordering a blood test (“Test ordered?”), obtaining blood from the patient (“Obtain sample”), completing examination of the blood sample (“Lab examination complete”), determining the results of the blood test in the laboratory (“Results generated”), informing healthcare personnel treating the patient of the results of the blood test (“Results reported to staff”), and whether the blood test is completed (“Test complete”).

Returning to the description of the flowchart of the method 200 shown in FIG. 2, at 214, a determination is made as to wait times for completion of actions 502 and/or steps 702 of actions 502. The controller 102 can communicate with the devices 104 to determine wait times for completing various actions 502 or steps 702. Optionally, the devices 104 can report the wait times to the database 108 for storage therein, and the controller 102 can examine the database 108 to determine the wait times. The wait times can be input by operators of the devices 104, and/or can be estimates based on how many orders are pending at the different locations (e.g., laboratory, imaging, etc.) ahead of the order(s) for the patient associated with the GUI 500, 600, 700.

The controller 102 can direct the device(s) 106 to display wait times 704 that are determined, as shown in FIG. 7. For example, the GUI 700 can be updated to show that there currently is a wait time or delay of forty-five minutes before the laboratory can complete examination of the blood sample that was obtained. This wait time can be provided by the operator of the device 104B or estimated by the backlog of examinations waiting to be completed at the laboratory.

At 216, a determination is made as to whether any actions 502 or steps 702 of actions 502 of the healthcare workflow remain to be completed. The controller 102 can examine data stored in the database 108 representative of the actions 502 and steps 702 of the actions 502 in the workflow of the patient, as well as data in the database 108 indicating which of the actions 502 or steps 702 are complete. The devices 104 can be used to provide or update the data indicating which actions 502 or steps 702 need to be completed or are complete.

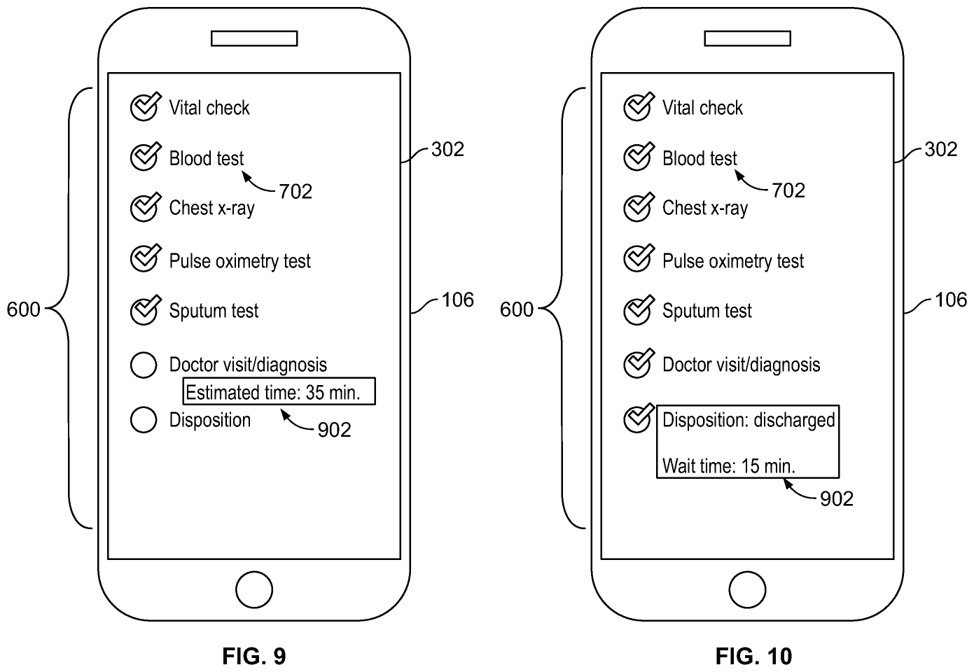

If any actions 502 or steps 702 remain to be completed, then flow of the method 200 can proceed toward 218, where the list of actions 502 and/or steps 502 to be completed shown on the device(s) 106 is updated. FIGS. 8 and 9 illustrate additional examples of updates to the GUI 600 that show completion of additional actions 502, as well as updated wait times 704 for not-yet-completed actions 502 or steps 702 (as determined at 214 and described above). The method 200 can proceed in a loop among 214, 216, 218 as the tests or examinations are completed, as wait times change, and/or as tests or examinations are added to or removed from the workflow.

In one embodiment, the controller 102 can automatically order, schedule, or otherwise arrange for a subsequent action 502 or step 702 to be performed following completion of a prior action 502 or step 702. For example, responsive to determining that the blood test is complete or that the sample for the blood test has been obtained, the controller 102 can schedule the chest x-ray for the patient using the imaging device 104C. This can cut down on delays between actions 502 or steps 702 that otherwise occur without the automatic scheduling of other actions 502 or steps 702.

Returning to the decision made at 216, once the actions 502 and steps 702 for disposition of the patient in the workflow are completed (as shown in the GUI 600 shown in FIG. 9), flow of the method 200 can proceed toward 220. At 220, a determination is made as to whether a disposition of the patient has been made. The disposition includes a decision by a healthcare personnel as to whether the patient is to be admitted to another healthcare facility, transferred to another healthcare facility, or discharged from the healthcare facility. For example, the disposition can include a decision by a treating physician as to whether the patient is to be admitted to a hospital for an extended stay of one or more nights, whether the patient is to be transferred to another hospital, or whether the patient is to be discharged and allowed to return home.

The controller 102 can examine data stored on the database 108 (e.g., from one or more of the devices 104) to determine whether the disposition of the patient has been determined. If the disposition has not been made, then flow of the method 200 can proceed toward 222. If the disposition has been made, then flow of the method 200 can proceed toward 226. At 222, a determination is made as to whether any additional actions 502 or steps 702 are needed before the healthcare personnel makes the disposition decision on the patient. For example, a doctor may request additional tests, examinations, consultations, or the like, before deciding whether to admit or discharge a patient. Optionally, the doctor making the disposition decision may be occupied due to other patients, a change in work schedule, or the like. The controller 102 can examine data stored in the database 108 to determine whether any additional actions 502 or steps 702 remain to be completed for the patient, and can direct the device(s) 106 to update the GUI to list the remaining actions 502 or steps 702.

At 224, the estimated time for completion of additional actions 502 or steps 702 before the disposition decision is made can be determined. The disposition decision for the patient may be delayed due to the unavailability of healthcare personnel, due to the need for additional actions 502 or steps 702, or the like. The controller 102 can examine estimated times to complete the actions 502 or steps 702, estimated times before the healthcare personnel is available to examine test results, etc., from the database 108 (e.g., as provided by one or more of the devices 104). The controller 102 can then direct the device(s) 106 to display an estimated wait time 902 for the healthcare personnel to arrive and make the disposition decision, as shown in the GUI 600 in FIG. 9.

The method 200 can proceed in a loop among 220, 222, 224 until a disposition of the patient is decided by healthcare personnel. Once the decision is made, the decision can be recorded in data stored in the database 108, and the controller 102 can determine that the decision has been made based on this data. Flow of the method 200 can then proceed toward 226.

At 226, the disposition decision on the patient is reported along with associated information. For example, the controller 102 can examine the data stored in the database 108 to determine whether the patient is being admitted, transferred, or discharged. The controller 102 also can examine data stored in the database 108 to determine an estimated wait time before the patient is admitted, transferred, or discharged. This information can be recorded into the database 108 by the charting device 104D, the registration device 104A, or another device 104. The controller 102 can direct the device(s) 106 to display the wait time 902 for the admission, transfer, or discharge of the patient, as shown in FIG. 10. This wait time 902 can be determined based on information provided by the registration device 104A or another device 104.

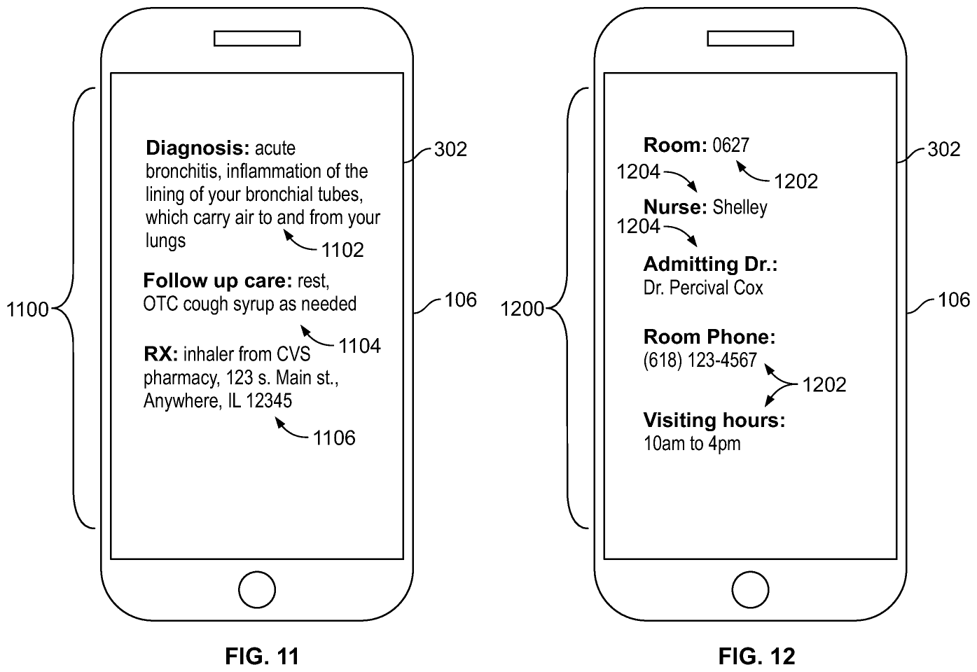

In one embodiment, the controller 102 can direct the device(s) 106 to display discharge information for the patient. FIG. 11 illustrates one example of a GUI 1100 showing discharge information for the patient. The GUI 1100 includes information such as a diagnosis 1102 of the patient, follow-up instructions 1104, and/or prescription information 1106. The diagnosis 1102 can be text and/or images that explain the medical issue or problem of the patient, as identified or determined by the healthcare personnel. The follow-up instructions 1104 can be instructions for how the patient is to care for himself or herself following departure from the healthcare facility. The prescription information 1106 can include information on what medications have been prescribed to the patient due to the diagnosis, as well as information on where the patient can obtain the prescribed medications. The controller 102 can obtain the information to be shown in the GUI 1100 from the database 108 (as provided by the devices 104) and can direct the device(s) 106 to present the information.

Optionally, the controller 102 can direct the device(s) 106 to display admission information for the patient. FIG. 12 illustrates one example of a GUI 1200 showing admission information for the patient. The GUI 1200 includes information such as a room information 1202 and personnel information 1204. The room information 1202 can indicate where the patient is being admitted, such as a room number, visiting hours, telephone number, address, etc. The personnel information 1204 can indicate the healthcare personnel that will be monitoring and/or treating the patient, such as a nurse and/or doctor currently assigned to monitor the patient. As the nurse and/or doctor end their respective work shift, the controller 102 can identify the nurse and/or doctor that is starting another work shift and can update the personnel information 1204 on the device(s) 106.

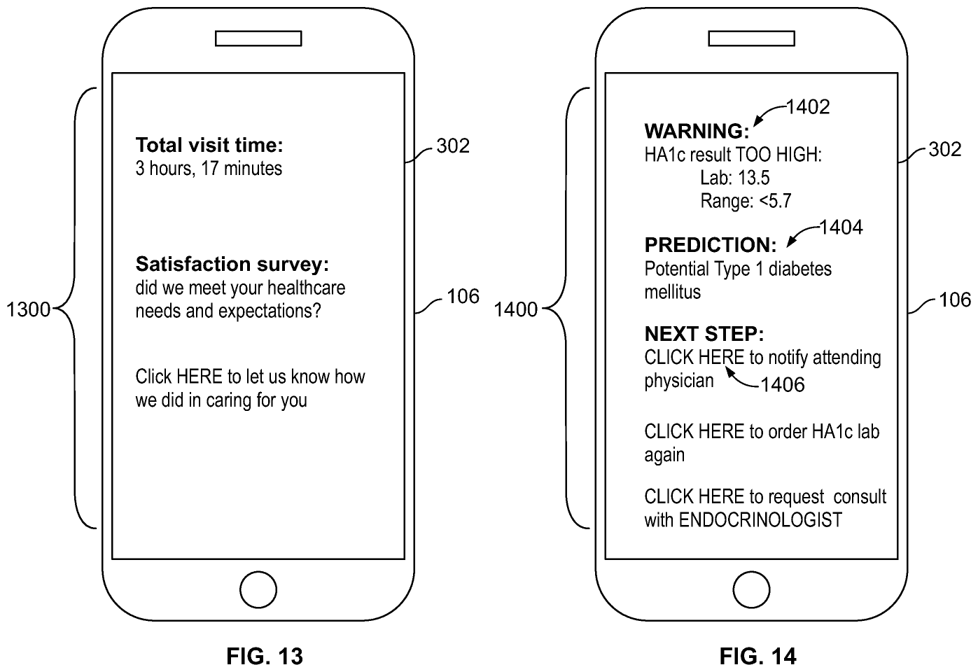

Returning to the description of the flowchart of the method 200 shown in FIG. 2, optionally, at 228, the satisfaction of the patient and/or approved persons can be queried. For example, the controller 102 can direct one or more of the devices 106 to present a summary GUI 1300, as shown in FIG. 13. This GUI 1300 can summarize the total visit time of the patient in the healthcare facility and can provide the patient and/or approved persons with a survey via the device(s) 106. This survey can provide a series of questions that measure how satisfied the patient and/or approved persons were with the visit to the healthcare facility. Because the systems and methods described herein provide clear and updated information on the status of the patient in the healthcare workflow, the patient and/or approved persons are more likely to be satisfied with the communication with the healthcare facility and are more likely to indicate (via the survey) their satisfaction with the healthcare facility. As described above, this can increase the financial awards or payments to, or reduce the fines of, the healthcare facility.

The tracking system 100 may alternatively or additionally provide updates, information, and/or predictions of health state to healthcare providers. For example, the tracking system 100 can provide notices and updates to nurses, doctors, veterinarians, nursing home staff, or the like, in addition to or in place of the information provided to friends, family members, and/or other approved persons (as described above). In one embodiment, the mobile device 106 of a healthcare provider can provide updates or statuses of various orders, lab requests, or the like, as described above in connection with FIGS. 7 through 9.

In one embodiment, the tracking system 100 can allow for healthcare providers to communicate with a medical facility before a patient is accepted or otherwise taken into the facility. For example, emergency medical services (EMS) personnel can use mobile devices 106 to communicate with the controller 102 at one or more facilities. The controller 102 can communicate with the devices 104 at each of the facilities and notify the mobile device(s) 106 of the EMS personnel of the capacity of the facilities to provide care to a patient being treated and/or transported by EMS personnel. For example, the devices 106 can communicate with the controller 102 to determine current wait times for patients, volumes of patients currently at the facilities, amounts of staff at the facilities (e.g., staffing ratios), or the like. This information can be used to determine which facility to take the patient to while also helping to spread out workloads and patient loads among facilities. This can help improve patient throughput at several facilities.

Optionally, the tracking system 100 can provide a warning or notification to the healthcare provider when a measured aspect of a patient is out of acceptable bounds or norms. FIG. 14 illustrates one example of a GUI 1400 presented on the device 106 that notifies a healthcare provider of a lab result. The controller 102 of the system 100 can direct the device 106 of the healthcare provider to generate a warning 1402 as or similar to that shown in the GUI 1400. This warning 1402 can be generated responsive to the controller 102 determining that a lab result (or other test or examination result) stored in the database 108 and/or from a data source device 104 has results that are out of acceptable bounds. For example, a lab result may indicate too high of a measured value of the patient (e.g., a cholesterol measurement greater than an upper defined limit), too low of a measured value (e.g., a blood pressure that is lower than a lower acceptable threshold), out of an acceptable range (e.g., a hemoglobin A1c result that is greater than an upper limit or lower than a lower limit), or the like.

The controller 102 can direct the device 106 of the healthcare provider to generate the warning 1402 so that the healthcare provider is made aware of the abnormal result sooner than current techniques and systems. For example, the healthcare provider does not need to wait for the lab results to be manually brought to the provider or electronically sent to the stationary computer of the healthcare provider. Instead, the healthcare provider is given the warning 1402 much faster, which can allow for the provider to take next steps to address the healthcare of the patient.

Optionally, the controller 102 can examine the lab, test, and/or examination results and provide a prediction 1404 to the healthcare provider via the device 106. For example, the database 108 can store a memory structure that associates different symptoms, test results, or the like, with different medical diagnoses. The controller 102 can examine information about the patient (e.g., provided by the registration device 104 and/or charting device 104D), test results of the patient (e.g., provided by the laboratory device 104B and/or charting device 104D), and/or other information, and compare this information to the memory structure in the database 108. If the combination of patient information and test results corresponds with a set of information associated with a diagnosis (in the database 108), then the controller 102 can direct the device 106 to generate the diagnosis prediction 1404 for the healthcare provider. The patient information stored in the database 108 may reveal recent weight loss, elevated blood sugar measurements, and/or an elevated hemoglobin A1c test result. The memory structure may associate this information (e.g., weight loss, high blood sugars, and/or high hemoglobin A1c result) with a diagnosis of Type I diabetes mellitus. As another example, symptoms of the patient entered into the database 108 by the charting device 104D and/or lab results generated by the laboratory device 104B may be associated in the memory structure with a sepsis diagnosis. This potential diagnosis can be provided to the healthcare provider by the controller 102 directing the device 106 to display an appropriate prediction 1404.

The controller 102 optionally can provide the healthcare provider with options for responsive actions to implement considering the prediction 1404. The database 108 can store several recommended actions 1406 associated with different diagnosis predictions 1404. For example, a prediction 1404 of type I diabetes mellitus can be associated with recommended actions 1406 to notify the attending physician, to order another lab test, to refer the patient to another physician, or the like. The controller 102 can obtain the recommended actions 1406 from the database 108 based on which diagnosis prediction 1404 was obtained. In the illustrated embodiment, the controller 102 directs the device 106 to provide clickable icons or text that allow the healthcare provider to automatically send an appropriate message to the attending physician (notifying the physician of the diagnosis prediction 1404), to automatically order the laboratory to run another test on the patient, and/or to automatically schedule a consultation for the patient with another type of physician based on the diagnosis prediction 1404. The healthcare provider can select one or more of these recommended actions 1406 on the device 106, and the controller 102 receives a signal indicating the selected action(s) 1406 from the device 106. The controller 102 may then send signals to direct a message be sent to another provider, to direct that another lab test be performed, or the like, based on which recommended action(s) 1406 was or were selected.

In one embodiment, a method is provided that includes identifying an electronic mobile device of one or more of a patient at a healthcare facility or at least one approved person selected by the patient, determining a current state of a healthcare workflow involving administration of healthcare services to the patient at the healthcare facility, directing the electronic mobile device to display information representative of the current state of the healthcare workflow for the patient, identifying one or more changes to the current state of the healthcare workflow, and directing the electronic mobile device to update the information that is displayed to correspond with the one or more changes to the current state of the healthcare workflow.

Optionally, the healthcare workflow includes a series of two or more tests, examinations, or medications to be administered to the patient. The healthcare workflow can include a series of actions to be performed in the administration of the healthcare services, and wherein the information that is displayed on the electronic mobile device indicates which of the actions have been completed and which of the actions are yet to be completed. The healthcare workflow can include a series of actions to be performed in the administration of the healthcare services, and the information that is displayed on the electronic mobile device indicates an estimated amount of time before a next action in the series of actions is to be completed.

The method also can include determining whether the patient is waiting for assignment to a treatment room while waiting in a waiting room, repeatedly notifying the patient (via the electronic mobile device) to check in with a healthcare personnel until the patient is assigned to the treatment room. Optionally, the method also includes directing the electronic mobile device to display a series of steps to complete at least one of the actions and updating display of the series of steps as one or more of the steps are completed.

The method can include additional operations of increasing and/or decreasing the actions included in the healthcare workflow based on an outcome of one or more of the actions (e.g., adding or removing designated actions from a designated or pre-defined list of actions in the workflow). The method also can include displaying a disposition notice on the electronic mobile device that indicates whether the patient is being admitted to another healthcare facility, transferred to another healthcare facility, or discharged from the healthcare facility.

Optionally, determining the current state of the healthcare workflow includes communicating with one or more electronic input devices of one or more of a healthcare facility registrar, a healthcare imaging facility, a healthcare laboratory facility, or a healthcare charting device. Directing the electronic mobile device to display the information representative of the current state of the healthcare workflow for the patient can include restricting access of the electronic mobile device to a subset of healthcare data stored in a centralized database that stores healthcare data for the patient and for additional patients. The method also can include providing the patient with a survey to measure satisfaction of the patient with the healthcare facility via the electronic mobile device. This survey can be provided at the patient is leaving the facility (e.g., at or near the completion of the healthcare workflow for that facility, even if the patient is being transferred to another facility).

In one embodiment, a system includes one or more processors of a communication controller configured to identify an electronic mobile device of one or more of a patient at a healthcare facility or at least one approved person selected by the patient. The one or more processors also are configured to determine a current state of a healthcare workflow involving administration of healthcare services to the patient at the healthcare facility, and to direct the electronic mobile device to display information representative of the current state of the healthcare workflow for the patient. The one or more processors also are configured to identify one or more changes to the current state of the healthcare workflow, and to direct the electronic mobile device to update the information that is displayed to correspond with the one or more changes to the current state of the healthcare workflow.

Optionally, the healthcare workflow includes a series of two or more tests, examinations, or medications to be administered to the patient. The healthcare workflow can include a series of actions to be performed in the administration of the healthcare services, and the information that is displayed on the electronic mobile device indicates which of the actions have been completed and which of the actions are yet to be completed. The healthcare workflow can include a series of actions to be performed in the administration of the healthcare services, and the information that is displayed on the electronic mobile device indicates an estimated amount of time before a next action in the series of actions is to be completed.

The one or more processors can be configured to determine whether the patient is waiting for assignment to a treatment room while waiting in a waiting room, and to repeatedly notify the patient, via the electronic mobile device, to check in with a healthcare personnel until the patient is assigned to the treatment room. The one or more processors can be configured to direct the electronic mobile device to display a series of steps to complete at least one of the actions, and to update display of the series of steps as one or more of the steps are completed.

Optionally, the one or more processors are configured to increase or decrease the actions included in the healthcare workflow based on an outcome of one or more of the actions. The one or more processors can be configured to direct the electronic mobile device to display a disposition notice on the electronic mobile device that indicates whether the patient is being admitted to another healthcare facility, transferred to another healthcare facility, or discharged from the healthcare facility. The one or more processors can be configured to determine the current state of the healthcare workflow by communicating with one or more electronic input devices of one or more of a healthcare facility registrar, a healthcare imaging facility, a healthcare laboratory facility, or a healthcare charting device.

The one or more processors can be configured to direct the electronic mobile device to display the information representative of the current state of the healthcare workflow for the patient by restricting access of the electronic mobile device to a subset of healthcare data stored in a centralized database that stores healthcare data for the patient and for additional patients. Optionally, the one or more processors are configured to direct the electronic mobile device to provide the patient with a survey to measure satisfaction of the patient with the healthcare facility via the electronic mobile device.

As used herein, an element or step recited in the singular and proceeded with the word “a” or “an” should be understood as not excluding plural of said elements or steps, unless such exclusion is explicitly stated. Furthermore, references to “one embodiment” of the presently described subject matter are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the subject matter set forth herein without departing from its scope. While the dimensions and types of materials described herein are intended to define the parameters of the disclosed subject matter, they are by no means limiting and are exemplary embodiments. Many other embodiments will be apparent to those of skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the subject matter set forth herein, including the best mode, and also to enable a person of ordinary skill in the art to practice the embodiments of disclosed subject matter, including making and using the devices or systems and performing the methods. The patentable scope of the subject matter described herein is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A method comprising:
identifying an electronic mobile device of one or more of a patient at a healthcare facility or at least one approved person selected by the patient;
determining a current state of a healthcare workflow involving administration of healthcare services to the patient at the healthcare facility;
directing the electronic mobile device to display information representative of the current state of the healthcare workflow for the patient;
identifying one or more changes to the current state of the healthcare workflow; and
directing the electronic mobile device to update the information that is displayed to correspond with the one or more changes to the current state of the healthcare workflow.

2. The method of claim 1, wherein the healthcare workflow includes a series of two or more tests, examinations, or medications to be administered to the patient.

3. The method of claim 1, wherein the healthcare workflow includes a series of actions to be performed in the administration of the healthcare services, and wherein the information that is displayed on the electronic mobile device indicates which of the actions have been completed and which of the actions are yet to be completed.

4. The method of claim 1, wherein the healthcare workflow includes a series of actions to be performed in the administration of the healthcare services, and wherein the information that is displayed on the electronic mobile device indicates an estimated amount of time before a next action in the series of actions is to be completed.

5. The method of claim 1, further comprising:
determining whether the patient is waiting for assignment to a treatment room while waiting in a waiting room;
repeatedly notifying the patient, via the electronic mobile device, to check in with a healthcare personnel until the patient is assigned to the treatment room.

6. The method of claim 1, further comprising:
directing the electronic mobile device to display a series of steps to complete one or more actions of the healthcare workflow; and
updating display of the series of steps as one or more of the steps are completed.

7. The method of claim 1, further comprising increasing or decreasing the actions included in the healthcare workflow based on an outcome of one or more of the actions.

8. The method of claim 1, further comprising displaying a disposition notice on the electronic mobile device that indicates whether the patient is being admitted to another healthcare facility, transferred to another healthcare facility, or discharged from the healthcare facility.

9. The method of claim 1, wherein determining the current state of the healthcare workflow includes communicating with one or more electronic input devices of one or more of a healthcare facility registrar, a healthcare imaging facility, a healthcare laboratory facility, or a healthcare charting device.

10. The method of claim 1, wherein directing the electronic mobile device to display the information representative of the current state of the healthcare workflow for the patient includes restricting access of the electronic mobile device to a subset of healthcare data stored in a centralized database that stores healthcare data for the patient and for additional patients.

11. The method of claim 1, further comprising providing the patient with a survey to measure satisfaction of the patient with the healthcare facility via the electronic mobile device.

12. A system comprising:
one or more processors of a communication controller configured to identify an electronic mobile device of one or more of a patient at a healthcare facility or at least one approved person selected by the patient, the one or more processors also configured to determine a current state of a healthcare workflow involving administration of healthcare services to the patient at the healthcare facility, and to direct the electronic mobile device to display information representative of the current state of the healthcare workflow for the patient,
wherein the one or more processors also are configured to identify one or more changes to the current state of the healthcare workflow, and to direct the electronic mobile device to update the information that is displayed to correspond with the one or more changes to the current state of the healthcare workflow.

13. The system of claim 12, wherein the healthcare workflow includes a series of two or more tests, examinations, or medications to be administered to the patient.

14. The system of claim 12, wherein the healthcare workflow includes a series of actions to be performed in the administration of the healthcare services, and wherein the information that is displayed on the electronic mobile device indicates which of the actions have been completed and which of the actions are yet to be completed.

15. The system of claim 12, wherein the healthcare workflow includes a series of actions to be performed in the administration of the healthcare services, and wherein the information that is displayed on the electronic mobile device indicates an estimated amount of time before a next action in the series of actions is to be completed.

16. The system of claim 12, wherein the one or more processors also are configured to determine whether the patient is waiting for assignment to a treatment room while waiting in a waiting room, and to repeatedly notify the patient, via the electronic mobile device, to check in with a healthcare personnel until the patient is assigned to the treatment room.

17. The system of claim 12, wherein the one or more processors are configured to direct the electronic mobile device to display a series of steps to complete at least one action of the healthcare workflow, and to update display of the series of steps as one or more of the steps are completed.

18. The system of claim 12, wherein the one or more processors are configured to increase or decrease the actions included in the healthcare workflow based on an outcome of one or more of the actions.

19. The system of claim 12, wherein the one or more processors are configured to direct the electronic mobile device to display a disposition notice on the electronic mobile device that indicates whether the patient is being admitted to another healthcare facility, transferred to another healthcare facility, or discharged from the healthcare facility.

20. A system comprising:
one or more processors of a communication controller configured to identify an electronic mobile device of one or more of a patient at a healthcare facility or at least one approved person selected by the patient, the one or more processors also configured to determine a current state of a healthcare workflow involving administration of healthcare services to the patient at the healthcare facility, and to direct the electronic mobile device to display information representative of the current state of the healthcare workflow for the patient,
wherein the one or more processors also are configured to identify one or more changes to the current state of the healthcare workflow, and to direct the electronic mobile device to update the information that is displayed to correspond with the one or more changes to the current state of the healthcare workflow,
wherein the healthcare workflow includes a series of two or more tests, examinations, or medications to be administered to the patient, and the healthcare workflow includes a series of actions to be performed in the administration of the healthcare services,
wherein the information that is displayed on the electronic mobile device indicates which of the actions have been completed and which of the actions are yet to be completed.
